(19) United States
(12) Patent Application Publication
(10) Pub. No.: US 2010/0112058 A1
(43) Pub. Date: May 6, 2010

(54) HYDROGEL MASK PACK, METHOD OF PREPARING THE SAME, AND RELATED COMPOSITION

(76) Inventors: Kun-Bin Lim, Gangwon-do (KR); Won Hur, Gangwon-do (KR); Ji-Young Kim, Gangwon-do (KR); Dong-Hyeon Lee, Gangwon-do (KR)

Correspondence Address: HARNESS, DICKEY & PIERCE, P.L.C., P.O. BOX 8910, RESTON, VA, 20195, US

(21) Appl. No.: 12/312,851

(22) PCT Filed: Jan. 11, 2007
(86) PCT No.: PCT/KR2007/000193

§ 371 Date: Dec. 29, 2009

(30) Foreign Application Priority Data
Dec. 14, 2006 (KR) 10-2006-0128076

(51) Int. Cl. A61K 8/64

(52) U.S. Cl. 424/484

(57) ABSTRACT

In a hydrogel mask pack having good skin-care effects, the hydrogel mask pack is formed by filling a frame with a composition for the hydrogel mask pack and immersing the frame in any one of an aqueous solution of a metal salt, alcohol or distilled water. The composition for the hydrogel mask pack is prepared by mixing an aqueous solution of fibroin and any one of alginate, cellulose or agar. The hydrogel mask pack may have superior moisturizing effects, skin-regenerative effects, mechanical strength and elasticity, and thus may be good enough to be used for a skin-care mask pack.

### TECHNICAL FIELD

Example embodiments of the present invention relate to a hydrogel mask pack, a method of forming the hydrogel mask pack and a composition used for the method. More particularly, example embodiments of the present invention relate to a hydrogel mask pack having skin-care effects and good physical properties, a method of forming the hydrogel mask pack and a composition used for the method.

### BACKGROUND ART

Generally, a mask pack for skin care has been formed using a nonwoven fabric in which an extract having skin-care effects, water or cosmetic water is included. However, when the cosmetic water or the extracts are added into the nonwoven fabric, the nonwoven fabric formed with a synthetic polymer may become stiff and the nonwoven fabric may not be pleasant to use.

When the nonwoven fabric is impregnated with a cosmetic composition in order to induce absorption of the cosmetic composition, the skin-adhesion properties of the nonwoven fabric may not be good and components included in the cosmetic composition may be exposed to air and thus degenerate. In addition, the components included in the cosmetic composition may stimulate the skin because the skin makes contact with the cosmetic composition having a high concentration at an early stage.

In order to overcome the above-mentioned problems, a hydrogel mask pack has been developed. Conventionally, hydrogel materials have been used that adhere to a nonwoven fabric, a mesh or a net. However, the hydrogel is often separated from the nonwoven fabric, the mesh or the net. When the hydrogel materials adhere to a synthetic polymer, such as the nonwoven fabric, many tests for biocompatibility and skin stability are required.

The hydrogel mask pack using natural materials has been developed overcome the above-mentioned problems of the mask pack. In the hydrogel mask pack using the natural materials, a film is formed using the natural materials without using the synthetic polymer such as the nonwoven fabric. Thus, side effects due to the synthetic polymer, such as itchiness, may not exist and the skin may be directly provided with nourishment.

Based on the above-mentioned problem, a mask pack using collagen has been commercialized. However, collagen is derived from animals. In addition, the mechanical strength of a film using collagen may not be strong enough to be used for the skin-care mask pack.

Silk fibroin has good biocompatibility to be used as a suture or a wound dressing. Examples of silk fibroin used for the wound dressing include a silk fibroin membrane for wound covering materials (Korean Patent Laid-Open Publication No. 2005-0051938), a wound dressing material containing silk fibroin and sericin as main components (Korean Patent Laid-Open Publication No. 2000-0068154) and a wound dressing of crystal fibroin (Korean Patent Laid-Open Publication No. 1998-0103243). The above-mentioned patent applications show that a film using silk fibroin has biocompatibility and also helps to heal injuries. However, the conventional fibroin film is not appropriate to use in a mask pack for skin care because the conventional fibroin film is a dry film without moisture, such as a wound dressing, and is intended to absorb moisture, such as that from a wound.

When a film is formed by gelling an aqueous solution of silk fibroin, the film may contain moisture. However, the film may be fragile and the mechanical strength of the film may not be substantially strong. Therefore, the conventional silk fibroin film is formed by methods such as drying, freeze-drying or alcohol treatment. As a result, the conventional film cannot be used for the mask pack for skin care.

Accordingly, there is a need for a method of forming a hydrogel mask pack using silk fibroin which has the high moisture content and the sufficient mechanical strength.

### DISCLOSURE OF THE INVENTION

### Technical Problem

Example embodiments of the present invention provide a composition used for forming a hydrogel mask pack having excellent skin-care effects and improved physical properties.

Example embodiments of the present invention provide a method of forming a hydrogel mask pack using the above-mentioned composition.

Example embodiments of the present invention provide a hydrogel mask pack formed by the above-mentioned method.

### Technical Solution

According to one aspect of the present invention, a composition for a hydrogel mask pack includes about 1 to about 10 percent by weight of fibroin, about 0.4 to about 4 percent by weight of any one of alginic acid, alginate, cellulose or agar and a remainder of water.

According to another aspect of the present invention, there is provided a method of forming a hydrogel mask pack. An aqueous solution of fibroin and any one of alginic acid, alginate, cellulose or agar are mixed to prepare a composition for the hydrogel mask pack. A frame is filled with the composition for the hydrogel mask pack. The frame is immersed in any one of an aqueous solution of a metal salt, alcohol or distilled water to gel the composition for the hydrogel mask pack.

In example embodiments of the present invention, the composition for the hydrogel mask pack may be centrifuged to extract a gel. The frame may be filled with the gel.

According to still another aspect of the present invention, there is provided a hydrogel mask pack. An aqueous solution of fibroin and any one of alginic acid, alginate, cellulose or agar are mixed to prepare a composition for the hydrogel mask pack. A frame is filled with the composition for the hydrogel mask pack. The frame is immersed in any one of an aqueous solution of a metal salt, alcohol or distilled water to form the hydrogel mask pack.

According to the present invention, the hydrogel mask pack may have improved moisturizing effects, good biocompatibility and prominent skin-regenerative effects by generating collagen. The hydrogel mask pack may also have superior physical properties, such as tensile strength, elongation rate and improved skin-adhesion properties. Therefore, the hydrogel mask pack in accordance with the present invention may be suitable for skin care.

### Effect of the Invention

According to the present invention, a hydrogel mask pack may have high moisture content, good moisturizing effects and good mechanical strength. Elongation rate and adhesion properties of the hydrogel mask pack may be good enough to be used for a skin-care mask pack. The hydrogel mask pack may have superior biocompatibility and skin-regenerative effects.

### BRIEF DESCRIPTION OF THE DRAWINGS

The above and other advantages of the present invention will become more apparent by describing in detail example embodiments thereof with reference to the accompanying drawings, in which:

FIG. 1 is a flowchart illustrating a method of forming a hydrogel mask pack in accordance with example embodiments of the present invention.

### BEST MODE FOR CARRYING OUT THE INVENTION

Hereinafter, the composition for the hydrogel mask pack, a method of forming the hydrogel mask pack using the composition, and the hydrogel mask pack formed by the method will be more fully described. The present invention may, however, be embodied in many different forms and should not be construed as limited to the example embodiments set forth herein. Rather, these embodiments are provided so that this disclosure will be thorough and complete, and will fully convey the scope of the present invention to those skilled in the art.

Composition for a Hydrogel Mask Pack

In accordance with the present invention, a composition for a hydrogel mask pack includes about 1 to about 10 percent by weight of fibroin, about 0.4 to about 4 percent by weight of any one of alginic acid, alginate, cellulose or agar and a remainder of water.

Fibroin is a kind of silk protein. In some example embodiments, “the silk protein” may be obtained from a silk-producing insect. For example, fibroin may be obtained from silk fiber from a silkworm.

The silk protein may include two strands of fibroin coated with sericin. Fibroin obtained from the silk protein has good biocompatibility and has no side effects on adjacent tissues. Fibroin also is hygroscopic, moisture-proof and moisture-permeable enough to be used for forming the hydrogel mask pack.

In some example embodiments, fibroin in the composition for the hydrogel mask pack may be obtained from raw silk fabric, a by-product of silk manufacture or a cocoon.

In some example embodiments, fibroin in the composition for the hydrogel mask pack may be dissolved in water to be used as an aqueous solution. For example, fibroin may be used after being dissolved in a salt solution, such as an aqueous solution of calcium chloride. Thereafter, the aqueous solution may be desalted by a dialysis membrane to obtain a pure aqueous solution of fibroin.

When the composition for the hydrogel mask pack includes less than about 1 percent by weight of fibroin, tensile strength of the mask pack formed using the composition may be less than about 0.1 kgf/cm², which is minimum tensile strength used for a skin-care mask pack. Therefore, the mask pack formed with the composition may not be used as the skin-care mask pack. In addition, a shrinkage may be increased in forming a mask pack in a frame.

When the composition for the hydrogel mask pack includes more than about 10 percent by weight of fibroin, it is difficult to mix additives with the composition and to fill the frame, because a viscosity of the composition may be high. Whereas the hydrogel mask pack formed using the composition may have substantially stronger mechanical strength, elasticity and softness of the mask pack may be deteriorated. Therefore, skin-adhesion properties may not be good.

The composition for the hydrogel mask pack includes about 1 to about 10 percent by weight of fibroin, and preferably about 4 to about 6 percent by weight of fibroin.

In some example embodiments, fibroin included in the composition for the hydrogel mask pack may have a weight average molecular weight of about 2,000 to about 400,000. The weight average molecular weight may be measured by dissolving fibroin in a salt solution.

When the weight average molecular weight is less than about 2,000, fibroin may not sufficiently form a gel and the composition may not be preferable for forming the hydrogel mask pack. When the weight average molecular weight of fibroin is more than about 400,000, the elasticity and elongation rate of the hydrogel mask pack may be deteriorated and the skin-adhesion properties may be degenerated.

Fibroin included in the composition for the hydrogel mask pack may have the weight average molecular weight of about 2,000 to about 400,000. Fibroin may preferably have the weight average molecular weight of about 50,000 to about 70,000 when considering the mechanical strength and the elasticity of the hydrogel mask pack formed using the composition.

In some example embodiments, alginic acid, alginate, cellulose, agar or a mixture thereof used in the composition for the hydrogel mask pack may serve to form the hydrogel mask pack having moisture and the strong mechanical strength.

When alginic acid, alginate, cellulose or agar makes contact with a metal salt, alcohol or distilled water, each of alginic acid, alginate, cellulose or agar may form a bond to have a hydrogel structure.

Alginic acid is a polymer including hundreds of β-1,4-linked mannuronic acid and L-guluronic acid. Alginic acid includes a carboxylic group. In an example embodiment, alginic acid may be used as a salt form. For example, the salt may be a sodium salt.

In accordance with the present invention, the composition includes about 0.4 to about 4 percent by weight of any one of alginic acid, alginate, cellulose or agar based on the total weight of the composition for the hydrogel mask pack.

When the composition includes less than about 0.4 percent by weight of any one of alginic acid, alginate, cellulose or agar, the viscosity of the composition may be low. Therefore, the hydrogel mask pack may not be formed using the composition. When the composition includes more than about 4 percent by weight of any one of alginic acid, alginate, cellulose or agar, the mechanical strength of the hydrogel mask pack may be improved. However, the viscosity of the composition may be excessively high and the softness and the elasticity of the hydrogel mask pack may be decreased to deteriorate fitting properties. Thus, the composition for the hydrogel mask pack includes about 0.4 to about 4 percent by weight of any one of alginic acid, alginate, cellulose or agar. The composition for the hydrogel mask pack may preferably include about 1 to about 3 percent by weight of any one of alginic acid, alginate, cellulose or agar in order to improve the mechanical strength and the skin-adhesion properties.

In some example embodiments, the composition for the hydrogel mask pack may include squalene or glycerin.

Squalene and glycerin are fat-soluble materials. Squalene and glycerin may supply the skin with oil and keep the skin from drying out. When squalene or glycerin is added in the composition for the hydrogel mask pack, the elongation rate of the hydrogel mask pack may be more increased and the mechanical strength of the hydrogel mask pack may be more decreased than those of the hydrogel mask pack formed using the composition which does not include squalene or glycerin.

Although the composition includes more than about 4 percent by weight of squalene or glycerin based on the weight of fibroin, the mechanical strength of the hydrogel mask pack may be kept almost constant compared to that of the hydrogel mask pack using the composition including about 4 percent by weight of squalene or glycerin. Thus, the amount of squalene or glycerin may be properly adjusted.

In some example embodiments, the composition for the hydrogel mask pack may include a moisturizer. Examples of the moisturizer may include any material that allows the hydrogel mask pack to maintain the proper mechanical strength, the elasticity and the skin-adhesion properties. Examples of the moisturizer may preferably include 1,3-butylene glycol, ethylene glycol, propylene glycol, hexylene glycol, sorbitol, mannitol, polyethylene glycol, polypropylene glycol, polyvinyl alcohol, hyaluronic acid, xanthan gum, acacia powder, etc. These may used alone or in a combination thereof. In order to improve the elongation rate, the composition for the hydrogel mask pack may preferably include polyethylene glycol or xanthan gum and more preferably include xanthan gum. When the composition for the hydrogel mask pack includes xanthan gum, physical properties and the elongation rate may be efficiently improved.

Squalene, glycerin or the moisturizer mentioned above are merely example embodiments of the present invention and the composition for the hydrogel mask pack may include other additional material having skin-care effects.

Hereinafter, a method of forming the hydrogel mask pack using the composition and the hydrogel mask pack formed by the method will be more fully described.

Hydrogel Mask Pack and a Method of Forming the Same

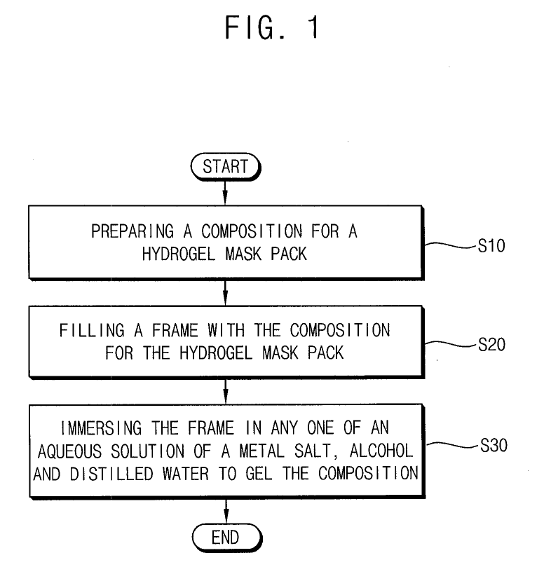

FIG. 1 is a flowchart illustrating a method of forming a hydrogel mask pack in accordance with example embodiments of the present invention.

Referring to FIG. 1, any one of alginic acid, alginate, cellulose or agar may be mixed with an aqueous solution of fibroin to prepare a composition for the hydrogel mask pack (Step S10). The composition for the hydrogel mask pack is previously described, so any further descriptions in this regard will be omitted herein.

A frame is filled with the composition for the hydrogel mask pack (Step S20). The frame is immersed in any one of an aqueous solution of a metal salt, alcohol or distilled water to gel the composition for the hydrogel mask pack (Step S30).

In some example embodiments, the composition for the hydrogel mask pack may be centrifuged to extract a gel. The frame may be filled with the gel.

When the composition for the hydrogel mask pack includes alginic acid or alginate, the frame filled with the composition may be immersed in the aqueous solution of the metal salt. When the composition for the hydrogel mask pack includes cellulose, the frame filled with the composition may be immersed in the alcohol. When the composition for the hydrogel mask pack includes agar, the frame filled with the composition may be immersed in the distilled water.

In some example embodiments, the metal salt may include a salt of an element from Group II or Group III in the periodic table. Examples of the element from Group II or Group III in the periodic table may include calcium, magnesium, barium, aluminum, etc. The metal salt may preferably include a calcium salt.

The hydrogel mask pack formed by the above-mentioned method may have high moisture content and also mechanical strength to be used for a skin-care mask pack.

In some example embodiments, the hydrogel mask pack may have the mechanical strength of about 0.1 kgf/cm², which is the preferable mechanical strength used for the skin-care mask pack.

When the moisture content of the hydrogel mask pack is measured by a moisture analyzer, the hydrogel mask pack may have about 90 percent by weight of the moisture content on average. Thus, the hydrogel mask pack may have good moisturizing effects as the skin-care mask pack.

The hydrogel mask pack may have superior skin-adhesion properties because elongation rate of the hydrogel mask pack may be good. The hydrogel mask pack may also closely adhere to and tighten the skin, and thus give the skin elasticity.

A shrinkage of the hydrogel mask pack due to dryness may not occur until after about 70 minutes of usage, and a condition of the hydrogel mask pack may be kept constant during usage of the hydrogel mask pack.

The hydrogel mask pack may remove sebum that is secreted excessively from the skin because the hydrogel mask pack may have a microporous structure and the good skin-adhesion properties.

In some example embodiments, the hydrogel mask pack may adjust a thickness according to a material and concentration of the material used for forming the hydrogel mask pack. The hydrogel mask pack may preferably have about 0.1 mm to about 3 mm of the thickness, and more preferably have about 0.3 mm to about 0.7 mm of the thickness.

In some example embodiments, the hydrogel mask pack may be formed using various types of frames and the hydrogel mask pack may be cut. As a result, the hydrogel mask pack may have various types of shapes according to adhesive areas such as a facial region.

In some example embodiments, the hydrogel mask pack may be used as a face mask pack, an eye mask pack or a nose mask pack.

The present invention will hereinafter be further described through Examples and Comparative Examples. Examples and Comparative Examples are illustrative of the present invention and are not to be construed as limiting thereof.

Synthesis of Aqueous Solution of Fibroin

### Synthesis Example 1

Raw silk fiber was washed in advance with hot water. An aqueous solution of sodium carbonate including the raw silk fiber was boiled for about 30 minutes at a temperature of about 100° C. The aqueous solution of sodium carbonate included about 0.3 g/100 mL of sodium carbonate. The aqueous solution of sodium carbonate including the raw silk fiber was washed with distilled water twice and was dried for 24 hours to obtain dried fibroin which corresponded to a central portion of silk fiber.

The dried fibroin was dissolved in small quantities in an aqueous solution of calcium chloride including about 37.5 g/100 mL of calcium chloride. Thus, a salt solution including fibroin was obtained. The salt solution included about 11 to about 12 percent by weight of fibroin. The fibroin dissolved in the salt solution had an average molecular weight of about 60,000.

In order to remove a high concentration of calcium chloride, a cellulose dialysis membrane by the name of Cellu.Sep, manufactured by Membrane Filtration Products Inc., in U.S.A., was prepared. The cellulose dialysis membrane was washed with distilled water twice and was kept in cold storage. The cellulose dialysis membrane had a molecular weight cut-off (MWCO) of about 6,000 to about 8,000 dalton.

After the cellulose dialysis membrane was filled with the salt solution including fibroin and was sealed, the cellulose dialysis membrane was put into a 2 L beaker filled with second distilled water. The second distilled water in the 2 L beaker was stirred using a magnetic bar. The salt was allowed to flow out from the dialysis membrane and the fibroin remained in the dialysis membrane. When a salt concentration of the distilled water measured by a refractometer and a conductive meter reached an equilibrium, the distilled water was exchanged. Pure fibroin was obtained performing the dialysis until an electrical conductivity was less than about 300 μs/cm.

Synthesis of a Hydrogel Mask Pack

### Example 1

After distilled water was added in the aqueous solution of fibroin of

Synthesis Example 1, sodium alginate, manufactured by Junsei Chemical Co., Ltd., in Japan, was slowly added and was stirred to obtain a composition for a hydrogel mask pack. The composition included about 2 percent by weight of sodium alginate and about 4 percent by weight of fibroin based on the total weight of the composition for the hydrogel mask pack.

The composition for the hydrogel mask pack was centrifuged at about 4,000 rpm for about 10 minutes in a centrifuge tube. A frame of 10 cm×10 cm was filled with a gel that settled in the centrifuge and was flattened using a plastic bar. Thereafter, the frame was immersed in an aqueous solution of calcium chloride including about 3 g/100 mL of calcium chloride for about 5 minutes. A film formed in the frame was turned over using tweezers and was immersed again in the aqueous solution of calcium chloride for about 5 minutes to form the hydrogel mask pack.

### Example 2

A hydrogel mask pack was formed by performing substantially the same process as that of Example 1 except for the content of sodium alginate in the composition for the hydrogel mask pack. The composition for the hydrogel mask pack included about 4 percent by weight of sodium alginate instead of about 2 percent by weight of sodium alginate based on the total weight of the composition for the hydrogel mask pack.

### Example 3

A hydrogel mask pack was formed by performing substantially the same process as that of Example 1 except for the content of fibroin in the composition for the hydrogel mask pack. The composition for the hydrogel mask pack included about 5 percent by weight of fibroin based on the total weight of the composition for the hydrogel mask pack.

### Example 4

A hydrogel mask pack was formed by performing substantially the same process as that of Example 1 except that squalene, manufactured by Cognis GmbH, in U.S.A., was further added into the composition for the hydrogel mask pack after adding sodium alginate. The composition further included about 5 percent by weight of squalene based on the total weight of the composition for the hydrogel mask pack.

### Example 5

A hydrogel mask pack was formed by performing substantially the same process as that of Example 4 except that glycerin was added instead of squalene into the composition for the hydrogel mask pack. The composition for the hydrogel mask pack further included about 5 percent by weight of glycerin based on the total weight of the composition for the hydrogel mask pack.

### Example 6

A hydrogel mask pack was formed by performing substantially the same process as that of Example 1 except that agar and distilled water were used instead of sodium alginate and an aqueous solution of calcium chloride, respectively, in the composition for the hydrogel mask pack. The composition for the hydrogel mask pack included about 1.5 percent by weight of agar based on the total weight of the composition for the hydrogel mask pack.

### Example 7

A hydrogel mask pack was formed by performing substantially the same process as that of Example 1 except that cellulose and an aqueous solution of ethanol, manufactured by Sigma-Aldrich Co., in U.S.A., were used instead of sodium alginate and the aqueous solution of calcium chloride, respectively, in the composition for the hydrogel mask pack. The aqueous solution of ethanol had a concentration of about 50 percent by weight of ethanol. The composition for the hydrogel mask pack included about 2 percent by weight of cellulose based on the total weight of the composition for the hydrogel mask pack.

### Examples 8 to 12

Hydrogel mask packs were formed by performing substantially the same process as that of Example 1 except that each of polyethylene glycol, polyvinyl alcohol, hyaluronic acid, xanthan gum or acacia powder were further added after adding sodium alginate into each of the compositions for the hydrogel mask pack. Each of the compositions for the hydrogel mask pack included about 1 percent by weight of each of polyethylene glycol, polyvinyl alcohol, hyaluronic acid, xanthan gum or acacia powder based on the total weight of the composition for the hydrogel mask pack.

### Comparative Example 1

A fibroin film was formed by performing substantially the same process as that of Example 1 except that the composition without sodium alginate was used.

### Comparative Example 2

A fibroin film was formed by performing substantially the same process as that of Comparative Example 1 except that glycerin was further added into the composition for the fibroin mask pack. The composition for the fibroin mask pack included about 10 percent by weight of glycerin based on the total weight of the composition for the fibroin mask pack.

### Comparative Example 3

A fibroin film was formed by performing substantially the same process as that of Example 1 except that a composition including about 0.7 percent by weight of fibroin based on the total weight of the composition was used.

### Comparative Example 4

A fibroin film was formed by performing substantially the same process as that of Example 1 except that a composition including about 11.4 percent by weight of fibroin based on the total weight of the composition was used.

### Comparative Example 5

A fibroin film was formed by performing substantially the same process as that of Example 1 except that a composition including about 0.2 percent by weight of sodium alginate based on the total weight of the composition was used.

### Comparative Example 6

A fibroin film was formed by performing substantially the same process as that of Example 1 except that a composition including about 5.2 percent by weight of sodium alginate based on the total weight of the composition was used.

Ingredients of the compositions and the contents thereof used in Examples 1 to 12 and Comparative Examples 1 to 6 are described in the following Table 1. The contents of ingredients are represented by a percent by weight based on the total weight of the composition.

TABLE 1

| | | | | | | | Poly- | Poly- | Hyal- | | |
|---|---|---|---|---|---|---|---|---|---|---|---|
| | | Sodium | | | | | ethylene | vinyl | uronic | Xanthan | Acacia |
| | Fibroin | alginate | Agar | Cellulose | Squalene | Glycerin | glycol | alcohol | acid | gum | powder |
| Example 1 | 4 | 2 | — | — | — | — | — | — | — | — | — |
| Example 2 | 4 | 4 | — | — | — | — | — | — | — | — | — |
| Example 3 | 5 | 2 | — | — | — | — | — | — | — | — | — |
| Example 4 | 4 | 2 | — | — | 5 | — | — | — | — | — | — |
| Example 5 | 4 | 2 | — | — | — | 5 | — | — | — | — | — |
| Example 6 | 4 | — | 1.5 | — | — | — | — | — | — | — | — |
| Example 7 | 4 | — | — | 2 | — | — | — | — | — | — | — |
| Example 8 | 4 | 2 | — | — | — | — | 1 | — | — | — | — |
| Example 9 | 4 | 2 | — | — | — | — | — | 1 | — | — | — |
| Example 10 | 4 | 2 | — | — | — | — | — | — | 1 | — | — |
| Example 11 | 4 | 2 | — | — | — | — | — | — | — | 1 | — |
| Example 12 | 4 | 2 | — | — | — | — | — | — | — | — | 1 |
| Comparative | 4 | — | — | — | — | — | — | — | — | — | — |
| Example 1 | | | | | | | | | | | |
| Comparative | 4 | — | — | — | — | 10 | — | — | — | — | — |
| Example 2 | | | | | | | | | | | |
| Comparative | 0.7 | 2 | — | — | — | — | — | — | — | — | — |
| Example 3 | | | | | | | | | | | |
| Comparative | 11.4 | 2 | — | — | — | — | — | — | — | — | — |
| Example 4 | | | | | | | | | | | |
| Comparative | 4 | 0.2 | — | — | — | — | — | — | — | — | — |
| Example 5 | | | | | | | | | | | |
| Comparative | 4 | 5.2 | — | — | — | — | — | — | — | — | — |
| Example 6 | | | | | | | | | | | |

### Experiment 1

Measurement of Tensile Strength

The hydrogel mask packs formed according to Examples 1 to 7 were cut to prepare pieces of the hydrogel mask pack having a size of 1.2 cm×1.2 cm×0.04 cm. After the pieces were mounted in a universal tester machine by the name of Vitrodyne V-1000, manufactured by Liveco, Inc., in U.S.A., tensile strength was determined using an initial grip distance of about 10 mm and cross-head speed of about 100 μm/s. Measurements were repeated three times and mean values were used. The tensile strength was calculated from measured values using the following formula. The results are shown in Table 2.

Tensile strength (kg/cm²)=maximum load (kg)/cross-sectional area of samples (cm²)

### Experiment 2

Measurement of Moisture Content

The hydrogel mask packs formed according to Examples 1 to 7 were cut to prepare pieces of the hydrogel mask pack having a size of 1.2 cm×1.2 cm×0.04 cm.

Moisture content was measured using a moisture analyzer by the name of MX-50, manufactured by A&D Company, Ltd., in Japan. The moisture analyzer used a drying loss method. The results are shown in Table 2.

Moisture content (%) {(weight before dryness−weight after dryness)/weight before dryness}×100

### Experiment 3

Measurement of a Maximum Elongation Length

The hydrogel mask packs formed according to Examples 1 to 7 were cut to prepare pieces of the hydrogel mask pack having a size of 1.2 cm×1.2 cm×0.04 cm. After the pieces were mounted in a universal tester machine by the name of Vitrodyne V-1000 manufactured by Liveco, Inc., in U.S.A., a maximum elongation length was determined using an initial grip distance of about 10 mm and cross-head speed of about 100 μm/s. The maximum elongation length was determined measuring the lengths of the pieces when the pieces were broken. Measurements were repeated three times and mean values were used. The results are shown in Table 2.

### Experiment 4

Measurement of Drying Rate

The hydrogel mask packs formed according to Examples 1 to 7 were cut to prepare rectangular pieces of the hydrogel mask pack having an area of about 36 cm². Areas were measured every ten minutes until two hours had passed, and were then measured every thirty minutes after two hours had passed since the experiments started. Times during which the area was shrunk were measured. The measurements were performed at a temperature of about 23.1° C. and at a humidity of about 38%. The results are shown in Table 2.

TABLE 2

| | Tensile | Maximum | | |
|---|---|---|---|---|
| | Strength | Elongation | Moisture | Shrinkage |
| Examples | (kgf/cm²) | (cm) | Content (%) | (minutes) |
| Example 1 | 0.20 | 3.4 | 92.65 | 72 |
| Example 2 | 0.49 | 3.5 | 90.11 | 72 |
| Example 3 | 0.31 | 3.7 | 91.22 | 72 |
| Example 4 | 0.12 | 3.7 | 85.93 | 70 |
| Example 5 | 0.13 | 3.8 | 86.67 | 70 |
| Example 6 | 0.39 | 4.2 | 85.55 | 71 |
| Example 7 | 0.71 | 3.7 | 87.21 | 70 |
| Comparative | Fragile | Fragile | Small | — |
| Example 1 | | | | |
| Comparative | A film was | A film was | A film was | — |
| Example 2 | not formed | not formed | not formed | |
| Comparative | 0.07 | 2.1 | 92.23 | — |
| Example 3 | | | | |
| Comparative | 0.42 | 1.7 | 80.21 | — |
| Example 4 | | | | |
| Comparative | A film was | A film was | A film was | — |
| Example 5 | not formed | not formed | not formed | |
| Comparative | 0.52 | 2.2 | 87.45 | — |
| Example 6 | | | | |

Referring to the results of Table 2, the hydrogel mask packs formed according to Examples 1 to 7 had the mechanical strengths more than about 0.1 kgf/cm², which is minimum mechanical strength used for the mask pack, and also had the moisture content more than about 85 percent by weight. The time after which shrinkage occurred was about 70 minutes. Therefore, a condition of the hydrogel mask pack had not changed for about one hour after the hydrogel mask pack was used.

Thus, the hydrogel mask packs formed according to Examples 1 to 7 are more preferable to a conventional fibroin film because the hydrogel mask packs formed according to Examples 1 to 7 have the mechanical strength and the moisture content.

In Comparative Examples, when the composition included less than about 0.4 percent by weight of sodium alginate, a film was not formed. When the composition included more than about 4 percent by weight of sodium alginate, the mechanical strength was good. However, the mask pack did not have good elongation rate and good elasticity to deteriorate skin-adhesion properties.

When the composition included less than about 1 percent by weight of fibroin, the film was not used for the skin-care mask pack because the film did not have the mechanical strength. When the composition included more than about 10 percent by weight of fibroin, the mechanical strength was good. However, the elongation rate and the elasticity of the mask pack were poor to deteriorate skin-adhesion properties.

### Experiment 5

Tensile strengths and elongation rates of the hydrogel mask packs formed according to Examples 8 to 12 are shown in Table 3. The hydrogel mask packs were formed using the composition including additives. The tensile strength was determined by performing substantially the same process as that of Experiment 1. The elongation rate was represented as a percent ratio of a difference of a maximum elongation length and an initial length, with respect to the initial length.

TABLE 3

| | | Tensile Strength | Elongation |
|---|---|---|---|
| Examples | Type of Additive | (kgf/cm²) | Rate (%) |
| Example 1 | None (control) | 0.39 | 166 |
| Example 8 | Polyethylene glycol | 0.33 | 176 |
| Example 9 | Polyvinyl alcohol | 0.29 | 124 |
| Example 10 | Hyaluronic acid | 0.25 | 88.7 |
| Example 11 | Xanthan gum | 0.40 | 197 |
| Example 12 | Acacia powder | 0.25 | 97 |

As shown in Table 3, when the additives were included in the composition for the hydrogel mask pack, the mechanical strength was decreased. However, the mechanical strength of the hydrogel mask pack was more than about 0.1 kgf/cm² and the length was extended by about two times as long as an original length to be used for the skin-care mask pack. Thus, the composition for the hydrogel mask pack may include various additives.

### Experiment 6

Test of Moisturizing Effects

Moisturizing effects were examined for 20 adult subjects (10 men and 10 women) that were 24 to 59 years old. The hydrogel mask pack formed according to Example 1 was cut to prepare pieces of the hydrogel mask pack having a size of 5 cm×5 cm×0.04 cm. Collagen sheets having substantially the same size were prepared for controls. Weights of pieces of the hydrogel mask pack and the collagen sheets were measured. After the moisture content of the each of the faces was measured, the pieces of the hydrogel mask pack and the collagen sheets were attached onto a left cheek and a right cheek of each of the subjects, respectively. The pieces were covered with vinyls in order to inhibit evaporation of the moisture. After 30 minutes, the pieces were detached from the cheeks and the moisture content of the faces of the subject was again measured. The results are shown in Table 4. The results are a mean value of about 20 adult subjects.

TABLE 4

| Type | Hydrogel mask pack | Collagen sheet |
|---|---|---|
| Initial weight | 2.458 g | 2.912 g |
| Weight after 30 minutes | 2.249 g | 2.135 g |
| Initial moisture content | 34.4% | 32.8% |
| Moisture content after 30 minutes | 54.2% | 47.8% |

As shown in Table 4, decreases in the weight of the hydrogel mask pack formed according to Example 1 was less than that of the collagen sheet after having been attached to the cheeks for 30 minutes. Therefore, the hydrogel mask pack had a low amount of dryness. Changes of the moisturizing effects in the skin directly showed that the hydrogel mask pack had superior moisturizing effects than those of the collagen sheets.

### Experiment 7

Test of Improving Elasticity

Pieces of the hydrogel mask pack formed according to Example 1 and collagen sheets were prepared by the same process as that of Experiment 6. Subjects were substantially the same as those of Experiment 6.

The hydrogel mask packs and the collagen sheets were attached to the left cheek and the right cheek, respectively, for 30 minutes everyday. After the above-mentioned experiments had been performed for 14 days, the elasticity of the skin was measured using an elasticity sensor manufactured by Aram HUVIS Co., Ltd., in Korea. Measurements were repeated twice and two measurement values are reflected in the results. The results are a mean value of the measurement values of about 20 of the subjects. The results are shown in Table 5.

TABLE 5

| Type | Before use | Hydrogel mask pack | Collagen sheet |
|---|---|---|---|
| Elasticity | 50% | 65% | 55% |

As shown in Table 5, the hydrogel mask pack improves the elasticity by about 10 percent better than the collagen sheets.

### INDUSTRIAL APPLICABILITY

According to the present invention, a hydrogel mask pack may have high moisture content, good moisturizing effects and good mechanical strength. Elongation rate and adhesion properties of the hydrogel mask pack may be good enough to be used for a skin-care mask pack. The hydrogel mask pack may have superior biocompatibility and skin-regenerative effects.

1. A method of forming a hydrogel mask pack comprising:
mixing an aqueous solution of fibroin and any one selected from the group consisting of alginate, cellulose and agar to prepare a composition for the hydrogel mask pack;
filling a frame with the composition for the hydrogel mask pack; and
immersing the frame in any one selected from the group consisting of an aqueous solution of a metal salt, alcohol and distilled water to gel the to composition.

2. The method of claim 1, wherein filling the frame with the composition for the hydrogel mask pack comprises:
centrifuging the composition for the hydrogel mask pack to extract a gel; and
filling the frame with the gel.

3. The method of claim 1, wherein the metal salt comprises a salt of a metal of Group II or Group III in the periodic table.

4. The method of claim 1, wherein the metal salt comprises at least one selected from the group consisting of calcium salt, magnesium salt, barium salt and aluminum salt.

5. The method of claim 1, wherein fibroin has a weight average molecular weight of about 2,000 to about 400,000.

6. The method of claim 1, wherein the composition for the hydrogel mask pack comprises:
about 1 to about 10 percent by weight of fibroin;
about 0.4 to about 4 percent by weight of any one selected from the group consisting of alginate, cellulose and agar; and
a remainder of water.

7. The method of claim 1, wherein the composition for the hydrogel mask pack comprises:
about 4 to about 6 percent by weight of fibroin;
about 1 to about 3 percent by weight of any one selected from the group consisting of alginate, cellulose and agar; and
a remainder of water.

8. The method of claim 1, wherein the composition for the hydrogel mask pack further comprises squalene or glycerin.

9. The method of claim 1, wherein the composition for the hydrogel mask pack further comprises at least one selected from the group consisting of 1,3-butylene glycol, ethylene glycol, propylene glycol, hexylene glycol, sorbitol, mannitol, polyethylene glycol, polypropylene glycol, polyvinyl alcohol, hyaluronic acid, xanthan gum and acacia powder.

10. A hydrogel mask pack formed by filling a frame with a composition for the hydrogel mask pack and immersing the frame in any one selected from the group consisting of an aqueous solution of a metal salt, alcohol and distilled water, wherein the composition for the hydrogel mask pack is prepared by mixing an aqueous solution of fibroin and any one selected from the group consisting of alginate, cellulose and agar.

11. The hydrogel mask pack of claim 10, wherein the hydrogel mask pack has a thickness of about 0.1 mm to about 2 mm.

12. A composition for a hydrogel mask pack comprising:
about 1 to about 10 percent by weight of fibroin;
about 0.4 to about 4 percent by weight of any one selected from the group consisting of alginate, cellulose and agar; and
a remainder of water.
